(19) United States
(12) Patent Application Publication
(10) Pub. No.: US 2015/0335243 A1
(43) Pub. Date: Nov. 26, 2015

(54) Method and Device for Wireless Transmission of Acoustic Cardiac Signals

(71) Applicant: Bernd Assmann, Furth (DE)

(72) Inventor: Bernd Assmann, Furth (DE)

(21) Appl. No.: 14/720,696

(22) Filed: May 22, 2015

(30) Foreign Application Priority Data
May 22, 2014 (DE) 102014209806.8

(51) Int. Cl. A61B 5/00; A61B 5/02; A61B 19/00; A61B 5/0205; A61B 7/04

(52) U.S. Cl. CPC A61B 5/0017; A61B 5/0205; A61B 7/04; A61B 5/0004; A61B 19/5225; A61B 5/02028; A61B 5/7285; A61B 2019/5236; A61B 2019/5242; A61B 2562/0204

(57) ABSTRACT

A method for wireless transmission of acoustic cardiac signals during a medical imaging examination is provided. The acoustic cardiac signals are acquired at a sampling frequency using an optical microphone, and the sampling frequency spans one period duration. The wireless transmission of the acoustic cardiac signals is accomplished by a transmission device that includes a controller and a transmission unit. The transmission unit includes a signal modulation unit including a transmit diode and a receive diode. The method includes activating the transmit diode using the controller for a time interval including the activation time. The activation time is less than a period duration. The method includes emitting a signal using the transmit diode during the activation time. The emitted signal is optically modulated based on the acoustic cardiac signals. The method also includes acquiring the modulated signals using the receive diode during the activation time, and wirelessly transmitting the signals.

This application claims the benefit of DE 10 2014 209 806.8, filed on May 22, 2014, which is hereby incorporated by reference in its entirety.

### BACKGROUND

The present embodiments relate to wireless transmission of acoustic cardiac signals during a medical imaging examination.

A medical imaging session (e.g., a magnetic resonance imaging session) may include a plurality of transmit/receive cycles that are combined to produce an image using a postprocessing operation. In the case of regions of a patient's body that move due, for example, to the patient's heartbeat, the image acquisition for the individual cycles is to take place in the same phase of the movement. Trigger signals for the magnetic resonance imaging are derived from the bodily movement. The trigger signals specify a trigger time instant for initiating the image acquisition. In order to acquire image data of a cardiac region of the patient, the acquisition of the image data by the medical imaging device is to be synchronized to the R wave of an ECG signal of the patient in order to provide that the image data acquired at different times always relate to the same cardiac phase.

ECG signals are frequently subject to noise interference due to injections of gradients. For this reason, the cardiac sound is alternatively acquired also by an optical microphone. However, using wireless and/or battery-powered optical microphones (e.g., rechargeable) gives rise to the problem that devices of the type consume up to four times more electrical power than existing prior art ECG devices. Wireless optical microphones are therefore limited in most cases to an operating time of a few hours (e.g., three hours), and such devices must first be recharged before such devices may be used again.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method and device that enable a power-saving, wireless transmission of acoustic cardiac signals during a medical imaging examination are provided.

In one embodiment, a method for wireless transmission of acoustic cardiac signals during a medical imaging examination is provided. The acoustic cardiac signals are acquired at a sampling frequency by an optical microphone, and the sampling frequency spans one period duration. The wireless transmission of the acoustic cardiac signals is realized by a transmission device that has a control unit and a transmission unit. The transmission unit includes a signal modulation unit having a transmit diode and a receive diode. The method includes activating the transmit diode by the control unit for a time interval including the activation time. The activation time is less than a period duration. The method includes emitting a signal by the transmit diode during the activation time. The emitted signal is optically modulated based on the acoustic cardiac signals. The method also includes acquiring the modulated signals by the receive diode during the activation time, and wirelessly transmitting the signals.

One or more of the present embodiments advantageously enable a transmission time of the transmit diode to be significantly shortened and consequently the energy consumption of the transmission device to be reduced. If, for example, the activation time amounts to 50% of the period duration, the energy consumption of the transmit diode may be reduced by 50%. Owing to the reduced energy consumption of the transmission device, an operating time of the transmission device may be significantly increased, to the effect that, for example, the operating time of the transmission device may be extended to a whole day without interruption. In one embodiment, the transmit diode includes an infrared light-emitting diode (IR LED) having a power consumption of approximately 100 mA.

A control unit may be, for example, a unit having a processor. The control unit also includes control software and/or control programs that are stored in a memory unit and are executed by the processor unit for the purpose of controlling the individual units of the transmission device. In addition, the control unit may also include the memory unit. A signal modulation unit may be understood, for example, a unit that generates an electrical signal from an optical signal (e.g., from the optically acquired acoustic cardiac signal). IA signal (e.g., an infrared signal) is emitted by the transmit diode (e.g., an IR LED). The signal is modulated (e.g., overlaid) by the optically acquired acoustic cardiac signals. The modulated signal is subsequently received by the receive diode (e.g., an infrared photodiode), and an electrical signal is generated by the receive diode based on the received signal and output. The optical microphone may include a rechargeable battery-powered and/or disposable battery-powered optical microphone.

The period duration corresponds, for example, to a reciprocal value of the sampling frequency of the heart rate of a patient. The sampling frequency is equal to approximately 10 times the heart rate to provide that an adequate signal quality may be achieved by the sampling. Given a heart rate of approximately 35 Hz to 40 Hz, the sampling rate may amount to approximately 400 Hz. The duration of a corresponding period accordingly amounts to approximately 2500 ms. The transmit diode may be activated exclusively during the activation time and is in a passive and/or inactive operating state outside of the activation time.

A particularly power-saving transmission device may be realized if the activation time amounts at a maximum to 10% of the period duration. As a result hereof, a power saving of the transmission device amounts to at least 90% compared to a continuous activation of the transmit diode. In one embodiment, the activation time amounts at a maximum to 5% of the period duration or to a maximum of 3% of the period duration. In one embodiment, however, the activation time amounts to approximately 2% of the period duration. A power saving may therefore amount to at least 95% to 98% compared to a continuous activation of the transmit diode. Given a continuous rated current of approximately 100 mA for the transmit diode (e.g., for the IR LED), this therefore results in an average power consumption of approximately 2 mA to 5 mA.

In an advantageous development, after the activation time has elapsed, the transmit diode is switched into a passive and/or an inactive operating state. As a result hereof, the transmit diode may be available for a signal modulation exclusively during the activation time interval, and consequently, an operating time of the transmit diode during a sampling cycle may be significantly reduced. A passive and/or an inactive operating state of the transmit diode may be, for example, an operating state of the transmit diode in which the transmit diode emits no signal and is in a power-saving operating state.

The activation time may include a switching time. A sample-and-hold circuit of the transmission unit is activated for the switching time within the activation time. As a result hereof, a latest signal that is acquired by the receive diode may be stored for a signal processing operation arranged downstream of the sample-and-hold circuit. In addition, an undesirable overwriting of the stored signal value may be prevented, and the stored signal value may accordingly be available for an acquisition cycle. The sample-and-hold circuit may store the last signal value acquired and forwarded by the receive diode when the circuit is in a deactivated state. A current/voltage converter unit may be arranged between the receive diode and the sample-and-hold circuit so that a current signal acquired by the receive diode is converted into a voltage signal that is present at an input of the sample-and-hold circuit. In one embodiment, the activation time includes a delay time that precedes the switching time of the sample-and-hold circuit.

In a further embodiment, a signal processing operation is carried out by a signal processing unit of the transmission unit during a processing time that corresponds to a difference between the period duration and the activation time and directly follows on from the activation time. This enables a sufficiently large time interval to be available to the signal processing unit in order, for example, for a filter unit, such as a bandpass filter unit, of the signal processing unit to become tuned to the new signal value stored within the sample-and-hold circuit. The signal processing unit may include a filter unit, an amplifier unit, and an ADC unit. For example, signal components that lie outside the frequency range of the cardiac sound are filtered out by the filter unit. In this case, for example, signals having a frequency of less than 20 Hz or less than 25 Hz, originating, for example, from a respiration of the patient, are filtered out. Signals having a frequency of greater than 45 Hz, greater than 40 Hz, or greater than 35 Hz, which include, for example, noise signals from the microphone and/or higher-frequency gradient noises, may be filtered out in the process. Alternatively or in addition, alias effects in the signals may also be filtered out by the filter unit. The signal processing unit may be connected upstream of a wireless signal transmission so that a digital signal is present using the ADC unit for the wireless transmission.

In one embodiment, a transmission device having a control unit and a transmission unit is provided. The transmission unit includes a signal modulation unit having a transmit diode and a receive diode. The transmission device is configured for performing a method for wireless transmission of acoustic cardiac signals during a medical imaging examination. The acoustic cardiac signals are acquired at a sampling frequency by an optical microphone, and the sampling frequency spans one period duration. The method includes activating the transmit diode using the control unit for a time interval including an activation time. The activation time is less than the period duration. The method also includes emitting a signal using the transmit diode during the activation time. The emitted signal is optically modulated based on the acoustic cardiac signals. The method includes acquiring the modulated signals using the receive diode during the activation time, and wirelessly transmitting the signals.

By virtue of one or more of the present embodiments, a transmit time of the transmit diode may be significantly shortened, and consequently, the energy consumption of the transmission device may be reduced. Owing to the reduced energy consumption of the transmission device, an operating time of the transmission device may be significantly increased, to the effect that the operating time of the transmission device may be extended to a whole day without interruption.

The advantages of the transmission device according to one or more of the present embodiments essentially correspond to the advantages of the method according to one or more of the present embodiments for wireless transmission of acoustic cardiac signals, which have been explained in detail in the foregoing. Features, advantages or alternative variants cited in this connection may also be applied analogously to the other claimed subject matters, and vice versa.

In one embodiment, the control unit is configured for switching the transmit diode into a passive and/or an inactive operating state after the activation time has elapsed. As a result hereof, the transmit diode (e.g., the IR LED) may be available for a signal modulation exclusively during the activation time interval, and consequently, an operating time of the transmit diode during a sampling cycle may be significantly reduced. In addition, the power consumption of the transmission unit may be significantly reduced in this way.

A particularly power-saving transmission device may be realized if the activation time amounts at a maximum to 10% of the period duration. As a result hereof, a power saving of the transmission device amounts to at least 90% compared to a continuous activation of the transmit diode. In one embodiment, the activation time amounts at a maximum to 5% of the period duration or at a maximum to 3% of the period duration. In one embodiment, the activation time amounts to approximately 2% of the period duration. A power saving may therefore amount to at least 95% to 98% compared to a continuous activation of the transmit diode.

According to another embodiment, the transmission unit has a signal processing unit having a sample-and-hold circuit that is activated by the control unit during a time interval. The time interval is included within the activation time. As a result hereof, a latest signal that is acquired by the receive diode may be stored for a signal processing operation arranged downstream of the sample-and-hold circuit. In addition, an undesirable overwriting of the stored signal value may be prevented, and the stored signal value may accordingly be available for an acquisition cycle.

The signal processing unit may include a filter unit and an amplifier unit that are arranged such that the filter unit and the amplifier unit are connected downstream of the sample-and-hold circuit. By this, an advantageous signal filtering and/or signal amplification prior to a wireless signal transmission may be carried out. For example, signal components that lie outside the frequency range of the cardiac sound are filtered out by the filter unit. For example, signals having frequencies of less than 20 Hz, originating, for example, from a respiration of the patient, and/or signals having a frequency of greater than 45 Hz or greater than 40 Hz that are, for example, noise signals from the microphone, are filtered out in the process.

In one embodiment, the transmission unit includes a signal processing unit having an ADC unit, to the effect that the signal processing unit advantageously provides digital signals for the wireless signal transmission.

One or more of the present embodiments relate to a motion detection unit for detecting a cardiac motion during a medical imaging examination. The motion detection unit includes an optical microphone and a transmission device. The transmission device includes a control unit and a transmission unit. The transmission unit includes a signal modulation unit having a transmit diode and a receive diode. The transmission device is configured for performing a method for wireless transmission of acoustic cardiac signals. The acoustic cardiac signals are acquired at a sampling frequency by an optical microphone, and the sampling frequency spans one period duration. The method includes activating the transmit diode using the control unit for a time interval including an activation time. The activation time is less than the period duration. The method includes emitting a signal using the transmit diode during the activation time. The emitted signal is optically modulated based on the acoustic cardiac signals. The method includes acquiring the modulated signals using the receive diode during the activation time, and wirelessly transmitting the signals.

By virtue of one or more of the present embodiments, a transmit time of the transmit diode may be significantly shortened, and consequently, the energy consumption of the transmission device may be reduced, to the effect that the transmission device is available together with the medical imaging device in a functionally ready state for a long examination period (e.g., one day) without, for example, a recharging operation. The advantages of the motion detection unit according to one or more of the present embodiments essentially correspond to the advantages of the method according to one or more of the present embodiments for wireless transmission of acoustic cardiac signals, which have been explained in detail in the foregoing. Features, advantages or alternative variants cited in this connection may also be applied analogously to the other subject matters, and vice versa.

One or more of the present embodiments also relate to a medical imaging device having a motion detection unit for detecting a cardiac motion using an optical microphone and a transmission device. The transmission device includes a control unit and a transmission unit. The transmission unit includes a signal modulation unit having a transmit diode and a receive diode. The transmission device is configured for the purpose of performing a method for wireless transmission of acoustic cardiac signals. The acoustic cardiac signals are acquired at a sampling frequency using an optical microphone, and the sampling frequency spans one period duration. The method includes activating the transmit diode using the control unit for a time interval including an activation time. The activation time is less than the period duration. The method includes emitting a signal using the transmit diode during the activation time. The emitted signal is optically modulated based on the acoustic cardiac signals. The method includes acquiring the modulated signals using the receive diode during the activation time, and wirelessly transmitting the signals.

By virtue of the embodiment according to one or more of the present embodiments, a transmit time of the transmit diode may advantageously be significantly shortened, and consequently, the energy consumption of the transmission device may be reduced, to the effect that the transmission device is available together with the medical imaging device in a functionally ready state and/or ready for operation for a long examination duration (e.g., one day) without additional charging operations. The advantages of the medical imaging device according to one or more of the present embodiments essentially correspond to the advantages of the method according to one or more of the present embodiments and the device according to one or more of the present embodiments for wireless transmission of acoustic cardiac signals, which have been explained in detail in the foregoing. Features, advantages or alternative variants cited in this connection may also be applied analogously to the other subject matters, and vice versa.

Cardiac imaging may be carried out on the patient using the medical imaging device. A cardiac motion is detected during a cardiac imaging session. The medical imaging device may include, for example, a magnetic resonance device, a computed tomography device, an AX-arm, etc.

### BRIEF DESCRIPTION OF THE DRAWINGS

FIG. 1 shows one embodiment of a medical imaging device having a motion detection unit in a schematic representation;

FIG. 2 shows one embodiment of a motion detection unit in a schematic representation;

FIG. 3 shows one embodiment of a method for wireless transmission of acoustic cardiac signals; and

FIG. 4 shows an exemplary timing diagram of a process flow of the method.

### DETAILED DESCRIPTION

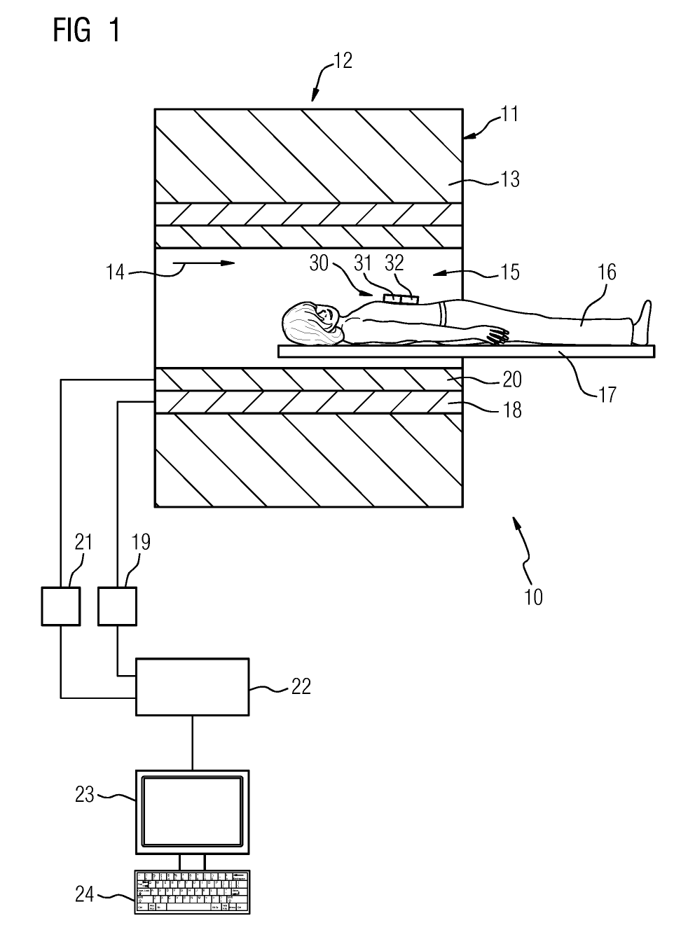

FIG. 1 shows one embodiment of a medical imaging device 10 in a schematic representation. In the present exemplary embodiment, the medical imaging device 10 is formed by a magnetic resonance device. In an alternative embodiment of the medical imaging device 10, the medical imaging device 10 may also be realized by a computed tomography device and/or a positron emission tomography (PET) device and/or other medical imaging devices 10 deemed beneficial by the person skilled in the art.

The magnetic resonance device includes a detector unit 11 that has a magnet unit 12 having a superconducting main magnet 13 for generating a strong and, for example, constant main magnetic field 14. The magnetic resonance device also includes a patient receiving zone 15 for accommodating a patient 16. In the present exemplary embodiment, the patient receiving zone 15 is embodied in a cylinder shape and is cylindrically enclosed by the magnet unit 12 in a circumferential direction. An alternative embodiment of the patient receiving zone 15 thereto may also be provided. The patient 16 may be introduced into the patient receiving zone 15 by a patient support device 17 of the magnetic resonance device.

The magnet unit 12 also includes a gradient coil unit 18 for generating magnetic field gradients that are used for spatial encoding during an imaging session. The gradient coil unit 18 is controlled by a gradient control unit 19 of the magnetic resonance device. The magnet unit 12 also includes a radiofrequency unit 20 and a radiofrequency antenna control unit 21 for exciting a polarization that becomes established in the main magnetic field 14 generated by the main magnet 13. The radiofrequency unit 20 is controlled by the radiofrequency antenna control unit 21 and radiates radiofrequency magnetic resonance sequences into an examination space that is substantially formed by the patient receiving zone 15 of the magnetic resonance device.

In order to control the main magnet 13, the gradient control unit 19, and the radiofrequency antenna control unit 21, the magnetic resonance device includes a system control unit 22 formed by a computing unit. The system control unit 22 is responsible for the centralized control of the magnetic resonance device, such as performing a predetermined imaging gradient echo sequence, for example. In addition, the system control unit 22 includes an evaluation unit (e.g., a processor; not shown in any further detail) for evaluating image data. Control information such as imaging parameters, for example, as well as reconstructed magnetic resonance images may be displayed for an operator on a display unit 23, for example, on at least one monitor of the magnetic resonance device. The magnetic resonance device also includes an input unit 24 by which information and/or parameters may be entered by an operator during a measurement process.

The magnetic resonance device includes a motion detection unit 30 for the purpose of detecting a cardiac motion of the patient 16. The motion detection unit 30 includes an optical microphone 31 for acquiring acoustic cardiac signals of the patient 16 and a transmission device 32 for wirelessly transmitting the acoustic cardiac signals acquired by the optical microphone 31. A cardiac motion during the medical imaging examination of the patient 16 is detected by the acoustic cardiac signals of the patient 16. In this way, trigger signals for initiating the imaging are generated by the motion detection unit 30 and/or the system control unit 22, thus providing that the acquired image data relates to an identical cardiac phase at all times.

In this case, the acoustic cardiac signals of the patient 16 are acquired at a sampling frequency by the optical microphone 31, where the sampling frequency spans one period duration Tₚₑᵣ. The sampling frequency amounts to approximately 400 Hz in order to provide that an adequate signal quality is obtained for the acquired acoustic cardiac signals. The resulting period duration Tₚₑᵣ of the sampling of the cardiac sound by the optical microphone 31 amounts to approximately 2500 μs. The frequencies of the cardiac sound lie in a range between approximately 25 Hz and approximately 35 Hz, up to a maximum of 40 Hz. The optical microphone 31 may include a rechargeable battery-powered and/or disposable battery-powered optical microphone 31.

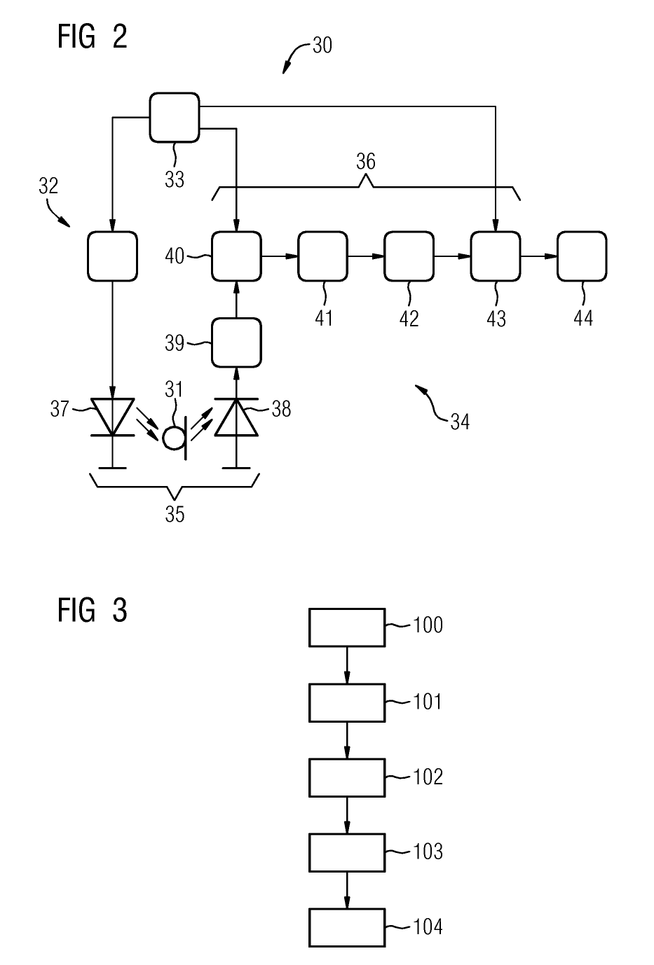

FIG. 2 shows one embodiment of the motion detection unit 30 in a schematic representation. The transmission device 32 of the motion detection unit 30 includes a control unit 33 (e.g., a controller) and a transmission unit 34. The control unit 33 is configured for controlling the transmission unit 34 of the transmission device 32. For this purpose, the control unit 33 includes a processor and corresponding control software and/or control computer programs that are stored in a memory unit and are executed in order to control the individual units of the transmission device 34 by the processor unit (e.g., the processor). The control unit 33 may also include the memory unit.

The transmission unit 34 of the transmission device 32 includes a signal modulation unit 35 and a signal processing unit 36. The signal modulation unit 36 includes a transmit diode 37 and a receive diode 38. The transmit diode 37 includes an infrared LED (IR LED) that emits a signal (e.g., an IR signal). The IR LED may include a continuous rated current of approximately 100 mA. The receive diode 38 includes an IR photodiode that is configured for acquiring the signals (e.g., IR signals) emitted by the transmit diode 37. After the IR signals have been emitted by the IR LED, the IR signals are optically modulated by the acoustic cardiac signals and subsequently received by the IR photodiode. The acoustic cardiac signals optically overlay the emitted IR signals. The modulated IR signals are acquired by the receive diode 38 (e.g., the IR photodiode) and converted into electrical signals (e.g., into analog electrical signals).

The signal processing unit 36 is arranged connected downstream of the signal modulation unit 35 inside the transmission unit 34. The signal processing unit 36 includes a current/voltage converter unit 39, a sample-and-hold circuit 40, a filter unit 41, an amplifier unit 42, and an ADC unit 43. The analog current signal of the receive diode 38 is converted into an analog voltage signal by the current/voltage converter unit 39. The sample-and-hold circuit 40 is configured for storing a latest signal acquired by the receive diode 38 for the further signal processing operation arranged downstream of the sample-and-hold circuit 40 and for providing the signal for the filter unit 41. The sample-and-hold circuit 40 may store the last signal value acquired and forwarded by the receive diode 38 when the circuit is in a deactivated state.

The filter unit 41 of the signal processing unit 36 serves to filter out alias effects in the signals for the downstream ADC unit 43. In addition, noise signals having frequencies below 20 Hz or below 25 Hz are filtered out of the signals by the filter unit 41. These noise signals are generated, for example, by low-frequency respiratory noises of the patient. Noise signals having frequencies greater than 45 Hz, greater than 40 Hz, or greater than 35 Hz are also filtered out of the signals by the filter unit 41. Noise signals of the type are produced, for example, by higher-frequency gradient noises and/or by rustling noises from the microphone. An output signal of the filter unit 41 accordingly has a frequency range of approximately 25 Hz to 35 Hz. In the present exemplary embodiment, the filter unit 41 is formed by a bandpass filter unit.

The amplifier unit 42 and the ADC unit 43 are arranged connected downstream of the filter unit 41 inside the signal processing unit 36. The filtered signals are amplified by the amplifier unit 42. The analog signals are converted into digital signals by the ADC unit 43 and are subsequently transmitted wirelessly by a transmit element 44 of the transmission unit 34 to a receive element (not shown in any further detail), which may be incorporated in the transmission device 32 and/or the system control unit 22.

FIG. 3 shows one embodiment of a method for wireless transmission of acoustic cardiac signals that have been acquired by the optical microphone 31. The method is performed by the transmission device 32. The individual units of the transmission device 32 are controlled for this purpose by the control unit 33 of the transmission device 32. In preparation for the performance of the method, the patient 16 is already positioned on the patient support device 17 and arranged together with the patient support device 17 inside the patient receiving zone 15.

In act 100, the transmit diode 37 formed by the IR LED is activated by the control unit 33 for a time interval including an activation time Tₐₖ. The activation time Tₐₖ is less than the period duration Tₚₑᵣ of the sampling of the cardiac sound using the optical microphone 31. The IR LED emits IR signals exclusively during the activation time Tₐₖ. The activation time Tₐₖ for the IR LED is aligned by the control unit 33 to the sampling, such that the activation time Tₐₖ of the IR LED coincides with and/or overlaps a time interval of an acoustic cardiac signal acquisition by the optical microphone 31.

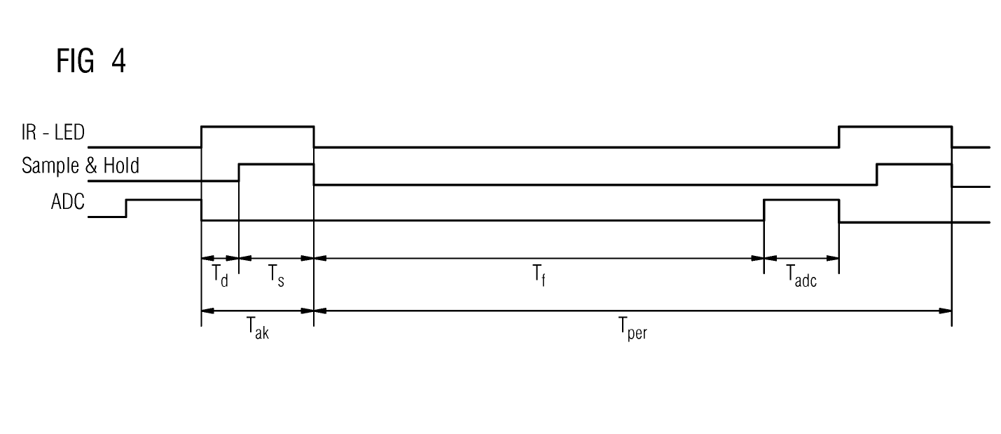

The activation time Tₐₖ of the IR LED amounts at a maximum to 10% of the period duration Tₚₑᵣ of 2500 μs (e.g., to 5% of the period duration Tₚₑᵣ or to approximately 2% of the period duration Tₚₑᵣ, in other words, to approximately 50 μs). Owing to the reduction in the operating time of the transmit diode 37 to approximately 50 μs, a power saving of approximately 98% is produced for the transmit diode 37 (FIG. 4). Accordingly, the transmit diode 37 (e.g., the IR LED) has only an average current consumption of 2 mA instead of the 100 mA.

In act 101, a signal that in the present exemplary embodiment is formed by an IR signal is emitted. The IR signal is emitted by the IR LED within the activation time Tₐₖ. In the process, the emitted IR signal is optically modulated based on the acoustic cardiac signals. For example, the acoustic cardiac signals optically overlay the emitted IR signals in this case.

After the IR signals have been emitted, the modulated IR signals are acquired during the activation time Tₐₖ in a further method act 102 by the receive diode 38 formed by the IR photodiode. In the act 102, an analog electrical output signal dependent on the acquired IR signal is generated by the receive diode 38.

Subsequently, in act 103, the analog electrical signals are processed further by the signal processing unit 36 and are transmitted wirelessly in a further method act 104 by the transmit element 44 of the transmission unit 34.

The signal processing method act 103 is performed partly during the activation time Tₐₖ and partly after the activation time Tₐₖ. Initially, in the signal processing method act 103, for example, a signal is converted from a current signal into a voltage signal by the current/voltage converter unit 39. The voltage signals are then forwarded to the sample-and-hold circuit 40 and stored at the sample-and-hold circuit 40. The sample-and-hold circuit 40 is activated by the control unit 33 for a switching time Tₛ. The switching time Tₛ is included in the activation time Tₐₖ (FIGS. 3 and 4). The activation time Tₐₖ also includes a delay time T_{d} that precedes the switching time Tₛ, such that the sample-and-hold circuit 40 is activated by the control unit 33 only after the delay time T_{d}. The switching time Tₛ ends essentially simultaneously with the activation time Tₐₖ.

Following the termination of the activation time Tₐₖ, the transmit diode 37 (e.g., the IR LED) is switched by the control unit 33 into a passive and/or inactive operating state. The transmit diode 37 (e.g., the IR LED) remains in the passive and/or inactive operating state until such time as the next sampling cycle for acquiring the acoustic cardiac signals by the optical microphone 31 begins, and consequently, the transmit diode 37 is once again activated by the control unit 33.

The activation time Tₐₖ is directly followed by a processing time Tₛᵥ. The processing time Tₛᵥ and the activation time Tₐₖ correspond in total substantially to the period duration Tₚₑᵣ. The processing time Tₛᵥ is therefore a difference between the period duration Tₚₑᵣ and the activation time Tₐₖ. During the signal processing time Tₛᵥ, a signal processing operation is performed by the signal processing unit 36 of the transmission unit 34. The signal processing operation in this case includes a filtering of the signals, an amplification of the signals and a conversion (e.g., digitization) of the signals into digital signals.

The signals are filtered by the filter unit 41 during a filter time T_{f}. The filter time T_{f} takes up more than 70% of the processing time Tₛᵥ. This enables the filter unit 41 (e.g., the bandpass filter unit) to become tuned to the signal value stored within the sample-and-hold circuit 40. The filter time T_{f} is directly followed by the ADC time T_{adc}. The filter time T_{f} and the ADC time T_{adc} together substantially correspond to the processing time Tₛᵥ. The ADC time T_{adc} essentially includes the amplification of the filtered signals by the amplifier unit 42 and the conversion (e.g., digitization) of the signals into digital signals by the ADC unit 43 (FIGS. 3 and 4).

Following the conversion (e.g., digitization) of the signals, the wireless transmission of the signals is accomplished by the transmit element 44 during method act 104. Trigger signals for initiating the acquisition of image data are generated by the motion detection unit 30 and/or the system control unit 22 based on the transmitted signals, to the effect that the individual sets of image data always relate to an identical motion phase of the heart of the patient 16.

Although the invention has been illustrated and described in greater detail based on the exemplary embodiments, the invention is not limited by the disclosed examples and other variations can be derived herefrom by the person skilled in the art without leaving the scope of protection of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for wireless transmission of acoustic cardiac signals during a medical imaging examination, wherein the acoustic cardiac signals are acquired at a sampling frequency using an optical microphone, and the sampling frequency spans one period duration, wherein the wireless transmission of the acoustic cardiac signals is accomplished using a transmission device comprising a controller and a transmission unit, wherein the transmission unit comprises a signal modulation unit comprising a transmit diode and a receive diode, the method comprising:
activating, by the controller, the transmit diode for a time interval including an activation time, the activation time being less than the period duration;
emitting, by the transmit diode, a signal during the activation time, the emitted signal being optically modulated based on the acoustic cardiac signals;
acquiring, by the receive diode, the modulated signals during the activation time; and
wirelessly transmitting the signals.

2. The method of claim 1, wherein the activation time amounts at a maximum to 10% of the period duration.

3. The method of claim 1, wherein the activation time amounts at a maximum to 5% of the period duration.

4. The method of claim 1, further comprising switching the transmit diode into a passive operating state, an inactive operating state, or a passive and inactive operating state after the activation time has elapsed.

5. The method of claim 1, wherein the activation time includes a switching time, and
wherein a sample-and-hold circuit of the transmission unit is activated for the switching time within the activation time.

6. The method of claim 1, further comprising performing a signal processing operation using a signal processing unit of the transmission unit during a processing time that corresponds to a difference between the period duration and the activation time, the processing time directly following the activation time.

7. A transmission device comprising:
a controller; and
a transmission unit comprising a signal modulation unit, the signal modulation unit comprising a transmit diode and a receive diode,
wherein the transmission device is configured to wirelessly transmit acoustic cardiac signals during a medical imaging examination, the acoustic cardiac signals being acquired at a sampling frequency using an optical microphone, the sampling frequency spanning one period duration, the wireless transmission of the acoustic cardiac signals comprising:
activation, by the controller, of the transmit diode for a time interval including an activation time, the activation time being less than the period duration;
emission, by the transmit diode, of a signal during the activation time, the emitted signal being optically modulated based on the acoustic cardiac signals;
acquisition, by the receive diode, of the modulated signals during the activation time; and
wireless transmission of the signals.

8. The transmission device of claim 7, wherein the controller is configured for switching the transmit diode into a passive operating state, an inactive operating state, or a passive and inactive operating state after the activation time has elapsed.

9. The transmission device of claim 7, wherein the activation time amounts at a maximum to 10% of the period duration.

10. The transmission device of claim 7, wherein the transmission unit comprises a signal processing unit, the signal processing unit comprising a sample-and-hold circuit that is activated by the controller during a time interval of a switching time, and
wherein the switching time is included in the activation time.

11. A motion detection unit for detecting a cardiac motion during a medical imaging examination, the motion detection unit comprising:
an optical microphone; and
a transmission device comprising:
a controller; and
a transmission unit comprising a signal modulation unit, the signal modulation unit comprising a transmit diode and a receive diode,
wherein the transmission device is configured to wirelessly transmit acoustic cardiac signals during a medical imaging examination, the acoustic cardiac signals being acquired at a sampling frequency using the optical microphone, the sampling frequency spanning one period duration, the wireless transmission of the acoustic cardiac signals comprising:
activation, by the controller, of the transmit diode for a time interval including an activation time, the activation time being less than the period duration;
emission, by the transmit diode, of a signal during the activation time, the emitted signal being optically modulated based on the acoustic cardiac signals;
acquisition, by the receive diode, of the modulated signals during the activation time; and
wireless transmission of the signals.

12. The motion detection unit of claim 11, wherein the controller is configured for switching the transmit diode into a passive operating state, an inactive operating state, or a passive and inactive operating state after the activation time has elapsed.

13. The motion detection unit of claim 11, wherein the activation time amounts at a maximum to 10% of the period duration.

14. The motion detection unit of claim 11, wherein the transmission unit comprises a signal processing unit, the signal processing unit comprising a sample-and-hold circuit that is activated by the controller during a time interval of a switching time, and
wherein the switching time is included in the activation time.

15. A medical imaging device comprising:
a motion detection unit for detecting a cardiac motion during a medical imaging examination, the motion detection unit comprising:
an optical microphone; and
a transmission device comprising:
a controller; and
a transmission unit comprising a signal modulation unit, the signal modulation unit comprising a transmit diode and a receive diode,
wherein the transmission device is configured to wirelessly transmit acoustic cardiac signals during a medical imaging examination, the acoustic cardiac signals being acquired at a sampling frequency using the optical microphone, the sampling frequency spanning one period duration, the wireless transmission of the acoustic cardiac signals comprising:
activation, by the controller, of the transmit diode for a time interval including an activation time, the activation time being less than the period duration;
emission, by the transmit diode, of a signal during the activation time, the emitted signal being optically modulated based on the acoustic cardiac signals;
acquisition, by the receive diode, of the modulated signals during the activation time; and
wireless transmission of the signals.

16. The medical imaging device of claim 15, wherein the controller is configured for switching the transmit diode into a passive operating state, an inactive operating state, or a passive and inactive operating state after the activation time has elapsed.

17. The medical imaging device of claim 15, wherein the activation time amounts at a maximum to 10% of the period duration.

18. The medical imaging device of claim 15, wherein the transmission unit comprises a signal processing unit, the signal processing unit comprising a sample-and-hold circuit that is activated by the controller during a time interval of a switching time, and
wherein the switching time is included in the activation time.
